(19) United States
(12) Patent Application Publication
(10) Pub. No.: US 2023/0119594 A1
(43) Pub. Date: Apr. 20, 2023

(54) SYSTEM AND METHOD FOR MONITORING AND RECOMMENDING POSTURE TO A USER

(71) Applicant: Logitech Europe S.A., Lausanne (CH)

(72) Inventors: Virgile Hernicot, Lausanne (CH); Nicolas Chauvin, Lonay (CH); Laleh Makarem, Pully (CH); Joy Oppliger, Cortaillod (CH); Olivier Theytaz, Savigny (CH); Olivier Girard, Autigny (CH)

(21) Appl. No.: 17/501,306

(22) Filed: Oct. 14, 2021

(51) Int. Cl. A61B 5/00; G06K 9/00; G06T 7/73; A61B 5/11

(52) U.S. Cl. CPC A61B 5/6885; G06K 9/00335; G06T 7/73; A61B 5/1116; A61B 5/6891

(57) ABSTRACT

A system for evaluating a posture of a user operating a computing device can include one or more processors, a sensor suite configured to generate sensor data corresponding to a three-dimensional (3D) orientation of a user's body, and one or more machine-readable, non-transitory storage mediums that include instructions configured to cause the one or more processors to perform operations including: estimating the user's posture based on the sensor data from the sensor suite; receiving application data corresponding to an application that the user is interfacing with on the computing device; and generating a classification of the user's posture based on a comparison of the estimated posture with a plurality of posture types. The performed operations may further include determining a recommendation to modify and improve the user's posture based on the classification and the application data and generating a user-accessible output that corresponds to the recommendation.

### BACKGROUND

Computers are ubiquitous and a mainstay in residential, commercial, and industrial settings. A typical user interaction with the laptop or desktop type computer usually includes an office or home office environment with a user sitting at a desk viewing a screen and interfacing with one or more input devices (e.g., keyboard, computer mouse, speakers, webcam, etc.). Long term use in these types of office environments can cause significant health issues over time. For instance, injuries (e.g., back pain, carpal tunnel syndrome, etc.) have accounted to over $150B a year in lost work days in the United States alone.

Significant innovation in improving the ergonomics of the office environment has helped to mitigate these health risks. For instance, Logitech® computer mice and keyboards are configured to place a user's hands in a more neutral position to reduce wrist strain. Some chairs include specialist lumbar support and other innovations that can better support a user's back and reduce back pain that typically occurs with long term use. Some clothing and clothing accessories (e.g., body wraps, shoulder restraints) operate to help a user keep better posture while walking or sitting, but are often too restrictive for many users. Despite the many innovations in improving the ergonomics of office environments and mitigating many of the health problems associated with office-related work activity, better solutions are needed to improve user posture in the office environment and to do so in a less-intrusive manner.

### BRIEF SUMMARY

In certain embodiments, a system for evaluating a posture of a user operating a computing device includes one or more processors, a sensor suite configured to generate sensor data corresponding to a three-dimensional (3D) orientation of a user's body, and one or more machine-readable, non-transitory storage mediums that include instructions configured to cause the one or more processors to perform operations including: estimating the user's posture based on the sensor data from the sensor suite; receiving application data corresponding to an application that the user is interfacing with on the computing device; generating a classification of the user's posture based on a comparison of the estimated posture with a plurality of posture types; determining a recommendation to modify and improve the user's posture based on the classification and the application data; and generating a user-accessible output that corresponds to the recommendation. The instructions can be further configured to cause the one or more processors to perform operations including determining a vertical axis relative to the user, where estimating the user's posture is further based on the vertical axis relative to the user.

In some embodiments, the sensor suite includes at least one of: an optical sensor from an optical device configured to generate optical sensor data corresponding to the 3D orientation of the user's body; a pressure sensor from a piece of furniture configured to generate force sensor data corresponding to the 3D orientation of the user's body; or a motion sensor from a wearable device configured to generate motion sensor data corresponding to the 3D orientation of the user's body. The application data can correspond to at least one of a list applications including: a video gaming application on the computing device; a productivity application on the computing device; a video conferencing application on the computing device; or the like, as would be appreciated by one of ordinary skill in the art with the benefit of this disclosure. The plurality of posture types may include at least: a good posture and bad posture, wherein the bad posture is the user having at least one of: asymmetry; forward leaning; backward leaning; side leaning; elbows to a side; lateral bending of the user's head; rotation of the user's head around a neck axis; flexion and extension of the user's neck; rounded shoulders, desk too high, and more, as would be appreciated by one of ordinary skill in the art with the benefit of this disclosure.

Determining the recommendation to modify and improve the user's posture can include determining body posture modifications that cause the user to have good posture while operating the given application. The user-accessible output can include a video underlay showing a user's target posture juxtaposed against the user's current actual posture. In some cases, the user-accessible output includes an animated 2D or 3D avatar showing a user's target posture versus the user's current actual posture (which can also be an avatar, an image of the user, or other graphical representation). In some aspects, the instructions can further be configured to cause the one or more processors to perform operations including determining a posture quality score that corresponds to a good posture versus bad posture ratio (or vice versa) over a given period of time, wherein generating the user-accessible output further includes generating a graphical and/or audio representation of the posture quality score.

In further embodiments, a computer-implemented method comprises: receiving sensor data from a sensor suite, the sensor data corresponding to a 3D orientation of a user's body; estimating the user's posture based on the received sensor data; receiving application data corresponding to an application that the user is interfacing with on the computing device; generating a classification of the user's posture based on a comparison of the estimated posture with a plurality of posture types; determining a recommendation to modify and improve the user's posture based on the classification and the application data; and generating a user-accessible output that corresponds to the recommendation. The method can further include determining a vertical axis relative to the user, wherein estimating the user's posture is further based on the vertical axis relative to the user. The sensor suite can include at least one of: an optical sensor from an optical device configured to generate optical sensor data corresponding to the 3D orientation of the user's body; a pressure sensor from a piece of furniture configured to generate force sensor data corresponding to the 3D orientation of the user's body; a motion sensor from a wearable device configured to generate motion sensor data corresponding to the 3D orientation of the user's body, or the like, as further described below. In some aspects, the application data can correspond to at least one of a list applications including: a video gaming application on the computing device; productivity application on the computing device; or a video conferencing application on the computing device. The plurality of posture types can include: a good posture and bad posture, wherein the bad posture is the user having at least one of: asymmetry; forward leaning; backward leaning; side leaning; elbows to a side; lateral bending of the user's head; rotation of the user's head around a neck axis; flexion and extension of the user's neck; rounded shoulders, the user's desk being too high, and more, as would be appreciated by one of ordinary skill in the art with the benefit of this disclosure. The user-accessible output can include at least one of: a video underlay showing a user's target posture juxtaposed against the user's current actual posture; or an animated 2D or 3D avatar showing a user's target posture versus the user's current actual posture.

In certain embodiments, a non-transitory computer-program product tangibly embodied in a machine-readable non-transitory storage medium can include instructions configured to cause one or more processors to perform operations including: receiving sensor data from a sensor suite, the sensor data corresponding to a 3D orientation of a user's body; estimating the user's posture based on the received sensor data; receiving application data corresponding to an application that the user is interfacing with on the computing device; generating a classification of the user's posture based on a comparison of the estimated posture with a plurality of posture types; determining a recommendation to modify and improve the user's posture based on the classification and the application data; and generating a user-accessible output that corresponds to the recommendation. In some aspects, the instructions are further configured to cause one or more processors to perform operations including determining a vertical axis relative to the user, wherein estimating the user's posture is further based on the vertical axis relative to the user.

The sensor suite can include at least one of: an optical sensor from an optical device configured to generate optical sensor data corresponding to the 3D orientation of the user's body; a pressure sensor from a piece of furniture configured to generate force sensor data corresponding to the 3D orientation of the user's body; a motion sensor from a wearable device configured to generate motion sensor data corresponding to the 3D orientation of the user's body, or the like, as further described below. In some aspects, the application data can correspond to at least one of a list applications including: a video gaming application on the computing device; productivity application on the computing device; or a video conferencing application on the computing device. The plurality of posture types can include: a good posture and bad posture, wherein the bad posture is the user having at least one of: asymmetry; forward leaning; backward leaning; side leaning; elbows to a side; lateral bending of the user's head; rotation of the user's head around a neck axis; flexion and extension of the user's neck; rounded shoulders, the user's desk being too high, and more, as would be appreciated by one of ordinary skill in the art with the benefit of this disclosure. The user-accessible output can include at least one of: a video underlay showing a user's target posture juxtaposed against the user's current actual posture; or an animated 2D or 3D avatar showing a user's target posture versus the user's current actual posture.

This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings, and each claim.

The foregoing, together with other features and examples, will be described in more detail below in the following specification, claims, and accompanying drawings.

The terms and expressions that have been employed are used as terms of description and not of limitation, and there is no intention in the use of such terms and expressions of excluding any equivalents of the features shown and described or portions thereof. It is recognized, however, that various modifications are possible within the scope of the systems and methods claimed. Thus, it should be understood that, although the present system and methods have been specifically disclosed by examples and optional features, modification and variation of the concepts herein disclosed should be recognized by those skilled in the art, and that such modifications and variations are considered to be within the scope of the systems and methods as defined by the appended claims.

### BRIEF DESCRIPTION OF THE DRAWINGS

The features of the various embodiments described above, as well as other features and advantages of certain embodiments of the present invention will be more apparent from the following detailed description taken in conjunction with the accompanying drawings, in which:

FIG. 1 shows a user working in a typical office environment;

FIG. 2 shows a simplified block diagram of a system to operate input device, according to certain embodiments;

FIG. 3 is a simplified block diagram of a computing device, according to certain embodiments;

FIGS. 4A-4F show different posture types that can be used to determine a user's posture, according to certain embodiments;

FIG. 5 is a simplified flow chart showing aspects of a method for monitoring a recommending posture to a user, according to certain embodiments;

FIG. 6 shows a graphical user interface showing a video underlay that provides a visual reference of good posture as compared to a user's current posture, according to certain embodiments; and

FIG. 7 shows a graphical user interface showing an avatar-based representation of a user and a visual reference of good posture for comparison, according to certain embodiments.

Throughout the drawings, it should be noted that like reference numbers are typically used to depict the same or similar elements, features, and structures.

### DETAILED DESCRIPTION

Aspects of the present disclosure relate generally to technologies related to ergonomics, and more particularly to systems and methods for providing virtual posture coaching for a user, according to certain embodiments.

In the following description, various examples of providing virtual posture coaching for a user are described. For purposes of explanation, specific configurations and details are set forth in order to provide a thorough understanding of the embodiments. However, it will be apparent to one skilled in the art that certain embodiments may be practiced or implemented without every detail disclosed. Furthermore, well-known features may be omitted or simplified in order to prevent any obfuscation of the novel features described herein.

To following high level summary is intended to provide a basic understanding of some of the novel innovations depicted in the figures and presented in the corresponding descriptions provided below. Aspects of the invention relate to systems and method for providing virtual posture coaching for a user. Products like input devices (e.g., keyboards, mice, etc.) and furniture (e.g., desks, chairs) can be ergonomically designed to promote good posture. Ergonomics can relate to designing workplaces based on the limitations and physical abilities of workers and can help to improve the efficiency of one's work environment. Poor posture, in particular, can cause a multitude of health issues as it puts extra stress on joints and muscles, which can lead to chronic pain including musculoskeletal disorders (e.g., tendonitis, carpal tunnel); detrimental impact to muscles, blood vessels, nerves, ligaments, and tendons; poor circulation; digestive issues; back, neck, and shoulder pain; headaches, and jaw pain; diminished lung function; fatigue, and other medical issues. Although many of the embodiments described herein are related to a user at a desk, it should be understood that aspects of the invention can apply to sit/stand desk systems or in sonic cases in standing-only activities. One of ordinary skill in the art with the benefit of this disclosure would appreciate the many possible applications thereof.

Despite the many available ergonomically designed products in the market, users still tend to exhibit poor posture over short and long term use by way they lean on the chair and orient their bodies in non-ideal positions. It should be noted that different activities may call for a different amount of strict adherence to conforming to an ergonomic ideal. For instance, gaming applications (e.g., FPS, RTS, etc.) and productivity applications (e.g., word processing) may call more a more robust adherence to an ergonomic ideal than other applications (e.g., video communication/collaboration, media watching/streaming, etc.), which is further described below.

Aspects of the invention can analyze a user's posture to determine if the posture is good or bad, and make recommendations for the user to improve their posture. Recommendations can be made in real-time, periodically, in summary format (e.g., daily or weekly report of posture performance over intervals), or the like. The system may determine that the user's posture is bad if they have asymmetry (e.g., crossed feet, leaning on elbow, shoulders forward), they are leaning forward (e.g., screen too far, feet too far back, desk too low, etc.), leaning backwards (e.g., feet forwards, shoulders supported), elbows are to the side (e.g., desk too high, mouse too far away), rounded shoulders (e.g., boxer chest, desk too low, keyboard forward, etc.), or the like. The system can analyze the user's posture via any suitable sensor suite that may comprise of images sensor(s) (e.g., webcam), pressure sensors (e.g., chair), inertial measurement unit (IMU) (e.g., on clothing, furniture, wearables, etc.), motion sensors, or other suitable sensor type and/or combination thereof. In some aspects, recommendations may include video-based feedback that shows an image of the user and posture, and an underlying reference images showing how the user should be sitting in order to have good posture. The user can adjust their posture in real-time until the user's image and the reference image are sufficiently aligned. In some cases, feedback can be given in the form of an avatar representation of the user's posture in comparison to a good posture reference. In some aspects, a good posture reference can be static or in some cases dynamic based on a history of previous measurements of the user (e.g., incremental improvement may be promoted, which may not be at an ideal position, for the sake of improving posture over time). Any suitable method of recommendation can be used as would be appreciated by one of ordinary skill in the art with the benefit of this disclosure. As noted above, the user's activity may affect the recommendation as different activities may warrant different postures or parameters of what constitutes “good” (e.g., healthy) posture. For instance, an example of a good dynamic posture is a posture that distributes weight over a variety of tissues (e.g., muscles, joints, bones, etc.) to minimize overloading in any of them over time.

By way of example, some embodiments may comprise a system for evaluating a posture of a user operating a computing device that includes one or more processors, a sensor suite (e.g., one or more sensors to generate optical sensor data, force sensor data, motion sensor data, orientation data, etc.) configured to generate sensor data corresponding to a three-dimensional (3D) orientation of a user's body, and one or more machine-readable, non-transitory storage mediums that include instructions configured to cause the one or more processors to perform operations including: estimating the user's posture based on the sensor data from the sensor suite and a determined vertical axis relative to the user; receiving application data corresponding to an application that the user is interfacing with on the computing device (e.g., video gaming, productivity, video conferencing, etc.); generating a classification of the user's posture (e.g., good posture and bad posture including asymmetry, forward/backward leaning, side leaning, elbows to the side, rounded shoulders, and more) based on a comparison of the estimated posture with a plurality of posture types; determining a recommendation to modify and improve the user's posture based on the classification and the application data; and generating a user-accessible output that corresponds to the recommendation (e.g., video underlay showing a user's target posture based versus the user's current actual posture, an animated avatar showing a user's target posture versus the user's current actual posture, etc.). In some aspects, the system can determine a posture quality score that corresponds to a good posture versus bad posture ratio over a given period of time, where generating the user-accessible output further includes generating a graphical representation of the posture quality score. In certain embodiments, the posture quality score is determined by the ratio of bad posture over good posture and may also take into account the persistence of a bad posture over a period of time. For example, if a user changes from a bad posture to a good posture every minute during a span of one hour, that user may have a better score than someone who continuously exhibited bad posture for thirty minutes followed by thirty minutes of good posture. One of ordinary skill in the art with the benefit of this disclosure would appreciate the many modifications, variations, and alternative embodiments thereof.

It is to be understood that this high level summary is presented to provide the reader with a baseline understanding of some of the novel aspects of the present disclosure and a roadmap to the details that follow. This high level summary in no way limits the scope of the various embodiments described throughout the detailed description and each of the figures referenced above are further described below in greater detail and in their proper scope.

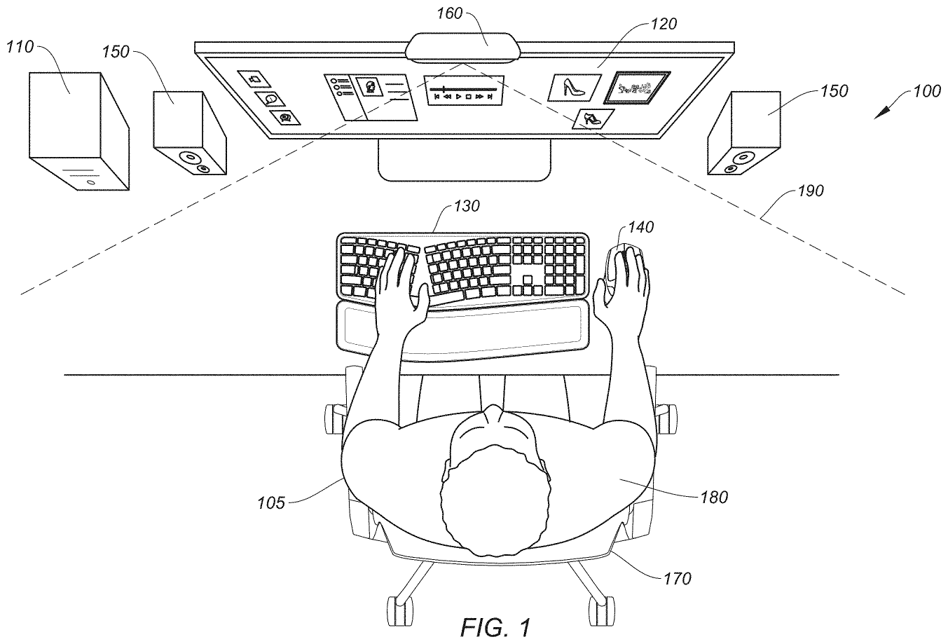

FIG. 1 shows an example of a computer system 100 that can include any of a variety of host computing devices and computer peripheral devices, according to certain embodiments. Computer system 100 shows a user 105 operating a host computing device (shown as a desktop computer) 110 and a number of computer peripheral devices communicatively coupled to and integrated with the host computing device, including a display device 120, a keyboard 130, a computer mouse 140, audio devices 150, webcam 160 with field of view 190, a smart chair 170 with force sensing and/or orientation detection capabilities, wearables 180 (e.g., shirt, watch, eye wear, etc.) with user movement and/or orientation detection capabilities, and any other suitable input devices (e.g., printer, headset, Wi-Fi hub, etc.) not shown. Each computer peripheral device 120-180 can be communicatively coupled to host computing device 110.

Although the host computing device is shown as a desktop computer, other types of host computing devices can be used including gaming systems, laptop computers, set top boxes, entertainment systems, tablet or “phablet” computers, stand-alone head mounted displays (“HMD”), or any other suitable host computing device (e.g., smart phone, smart wearable, or the like). In some cases, multiple host computing devices may be used and one or more of the computer peripheral devices may be communicatively coupled to one, some, or all of the host computing devices (e.g., a computer keyboard may be coupled to multiple host computing devices and may switch between them using Flow™ technology from Logitech®). A host computing device may also be referred to herein as a “host computer,” “host device,” “computing device,” “computer,” or the like, and may include a machine readable medium (not shown) configured to store computer code, such as driver software, firmware, and the like, where the computer code may be executable by one or more processors of the host computing device(s) to control aspects of the host computing device, for instance via the one or more computer peripheral devices.

A typical computer peripheral device can include any suitable input device, output device or input/output device including those shown (e.g., a keyboard) and not shown (e.g., remote control, wearables (e.g., gloves, watch, head mounted display), AR/VR controller, CAD controller, joystick, simulation shifter, stylus device, or other suitable device) that can be used, for example, to convert analog inputs into digital signals for computer processing. By way of example, a keyboard may be configured to provide control signals including button click events (e.g., corresponding to a pressing of one or more keys on the keyboard), audio signals (e.g., audio cues, integrated speakers), visual output signals (e.g., controlling one or more LEDs on the keyboard—controlled by the keyboard, the host computing devices coupled thereto, or a combination thereof), or the like. In another example, a computer peripheral device (e.g., computer mouse 140) can be configured to provide control signals for movement tracking (e.g., x-y movement on a planar surface, three-dimensional “in-air” movements, etc.), touch and/or gesture detection, lift detection, orientation detection (e.g., in 3 degrees-of-freedom (DOF) system, 6 DOF systems, etc.), power management capabilities, input detection (e.g., buttons, scroll wheels, etc.), output functions (e.g., LED control, haptic feedback, etc.), or any of myriad other features that can be provided by a computer peripheral device, as would be appreciated by one of ordinary skill in the art. In many of the embodiments described herein, one or more computer peripheral devices may include one or more sensors, such as optical sensors (e.g., webcam 160), motion sensors, force sensors (e.g., chair 170), or other sensor type, that would be appreciated by one of ordinary skill in the art with the benefit of this disclosure, that can be used to determine a user's posture. In certain embodiments, a computer peripheral device may include display device 120 (also referred to as a “monitor”) or other display device (e.g., mobile smart device) to provide posture feedback to the user, as further described below.

An input device may be a computer peripheral device, and may be referred to as either herein, as well as a “peripheral input device,” “peripheral,” or the like. In some cases, input devices may be referred to as human interface devices (HIDs) and their corresponding control signals may be referred to as HID commands. As noted above, the majority of the embodiments described herein generally refer to computer peripheral devices 120-170, however it should be understood that a computer peripheral device can be any suitable input/output (I/O) device (e.g., user interface device, control device, input unit, or the like) that may be adapted to utilize the novel embodiments described and contemplated herein.

### Typical System Embodiment for Operating a Computer Peripheral Device

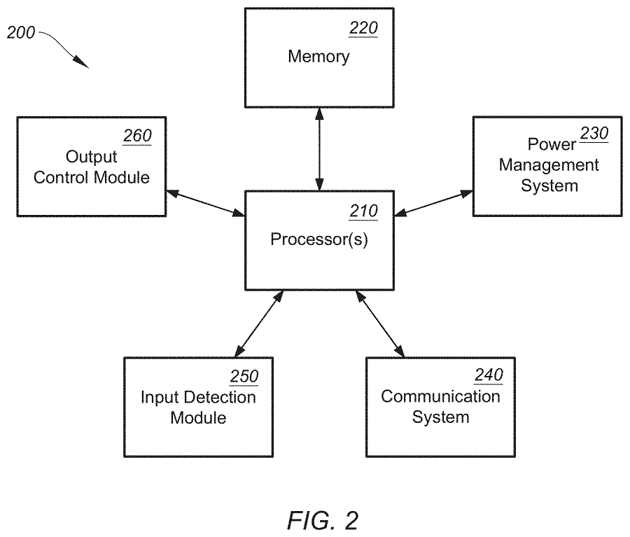

FIG. 2 shows a system 200 for operating a peripheral input device, according to certain embodiments. System 200 may be configured to operate any of the peripheral devices specifically shown and described herein (e.g., monitor 120, keyboard 130, mouse 140, speakers 150, etc.) or peripheral not shown (e.g., IoT devices, printer, etc.) but within the wide purview of the present disclosure. System 200 may include processor(s) 210, memory 220, a power management system 230, a communication system 240, an input detection system 250, and an output control system 260, each being alternatively referred to as “block” (e.g., memory block 220, power management system block 230, etc.). Each of the system blocks 220-260 can be in electrical communication with the processor(s) 210 (e.g., via a bus system). System 200 may also include additional functional blocks that are not shown, discussed, or necessarily germane to novelty to prevent obfuscation of the novel features described herein. System blocks 220-260 may be implemented as separate blocks, or alternatively, more than one system block may be implemented in a single block. In the context described herein, system 200 can be incorporated into any peripheral device described herein and may be configured to perform any of the various methods of providing posture feedback to a user including user posture detection (e.g., image sensor, force sensor, etc.) and user posture feedback (e.g., monitor 120, speakers 150, etc.), or the like, as would be appreciated by one of ordinary skill in the art with the benefit of this disclosure.

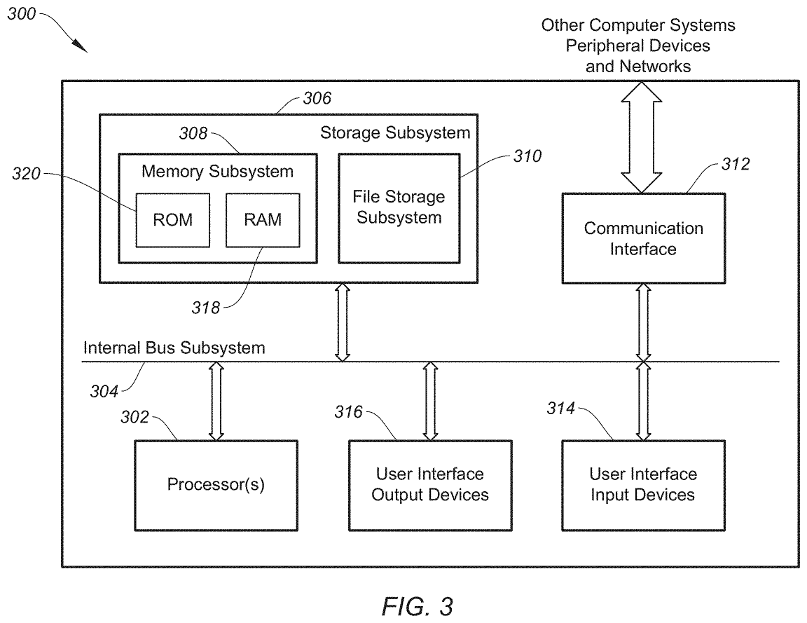

In certain embodiments, processor(s) 210 may include one or more microprocessors and can be configured to control the operation of system 200. Alternatively, processor(s) 210 may include one or more microcontrollers (MCUs), digital signal processors (DSPs), or the like, with supporting hardware and/or firmware (e.g., memory, programmable I/Os, etc.), as would be appreciated by one of ordinary skill in the art. Processor(s) 210 can control some or all aspects of operation of input device 130, 140, 160, 170 (e.g., system block 220-260). Alternatively or additionally, some of system blocks 220-260 may include an additional dedicated processor, which may work in conjunction with processor(s) 210. Processor(s) 210 may be local to the peripheral device (e.g., contained therein), may be external to the peripheral device (e.g., off-board processing, such as by a corresponding host computing device), or a combination thereof. As further described below, processor 302 of FIG. 3 may work in conjunction with processor 210 to perform some or all of the various methods (e.g., method 500) described throughout this disclosure. One of ordinary skill in the art would understand the many variations, modifications, and alternative embodiments that are possible. Processor(s) 210 may perform any of the various functions and methods described and/or covered by this disclosure, and may operate to generate the various commands (e.g., HID commands, etc., in conjunction with any other resources/blocks in system 200) and corresponding functions described herein.

Memory 220 can store one or more software programs to be executed by processors (e.g., in processor(s) 210). It should be understood that “software” can refer to sequences of instructions that, when executed by processing unit(s) (e.g., processors, processing devices, etc.), cause system 200 to perform certain operations of software programs. The instructions can be stored as firmware residing in read-only memory (ROM) and/or applications stored in media storage that can be read into memory for processing by processing devices. Software can be implemented as a single program or a collection of separate programs and can be stored in non-volatile storage and copied in whole or in-part to volatile working memory during program execution.

In some embodiments, memory 220 may store sensor data from any of the computer peripheral devices described herein, and particularly sensor data that can be used to determine a user's posture. Memory 220 can store data posture characterization data corresponding to an ideal posture and various representations of bad posture (e.g., asymmetry, forward/backward leaning, etc.), as further described below. Memory 220 can store user posture classification dataincluding real-time data and stored data, which can be used to generate real-time or batched (e.g., daily, weekly) reports to inform the user of their posture habits. Memory 220 can store avatar data or other graphical data that can be used to visually depict how a user's posture compares to an example of good posture and/or depict a user's posture quality score, as further described below. Memory 220 can be used to store any data described throughout this specification and particularly data that can be used to determine a user's posture, compare and classify/characterize a user's posture, and output information to a user to report the user's posture in any of a plurality of visual, audio, haptic, or other suitable formats, as further described herein and as would be appreciated by one of ordinary skill in the art with the benefit of this disclosure.

Power management system 230 can be configured to manage power distribution, recharging, power efficiency, haptic motor power control, and the like. In some embodiments, power management system 230 can include a battery (not shown), a USB based recharging system for the battery (not shown), and power management devices (e.g., voltage regulators—not shown). In certain embodiments, the functions provided by power management system 230 may be incorporated into processor(s) 210. The power source can be a replaceable battery, a rechargeable energy storage device (e.g., super capacitor, Lithium Polymer Battery, NiMH, NiCd), or a corded power supply. The recharging system can be an additional cable (specific for the recharging purpose) or it can use a USB connection to recharge the battery.

Communications system 240 can be configured to provide wireless communication with a corresponding host computing device or other devices and/or peripherals, according to certain embodiments. Communications system 240 can be configured to provide radio-frequency (RF), Wi-Fi, RFID, NFC, Bluetooth®, infra-red (IR), ZigBee®, or other suitable communication technology to communicate with other computing devices and/or peripheral devices. System 200 may optionally comprise a hardwired connection to the corresponding host computing device. For example, input device 130, 140, 160, 170 can be configured to receive a Universal Serial Bus (USB) cable to enable bi-directional electronic communication with the corresponding host computing device or other external devices. Some embodiments may utilize different types of cables or connection protocol standards to establish hardwired communication with other entities. In some aspects, communication ports (e.g., USB), power ports, etc., may be considered as part of other blocks described herein (e.g., input detection module 250, output control modules 260, etc.).

Input detection system 250 can control the detection of a user-interaction with input elements on the input device. For instance, input detection system 250 can detect user inputs from keys, buttons, roller wheels, scroll wheels, touch-sensitive touchpads, click wheels, dials, keypads, microphones, GUIs, touch-sensitive GUIs, image sensor based detection capable of user body tracking such as posture estimation, face tracking, gesture tracking (e.g., via webcam 160), audio based detection such as voice input (e.g., via microphone), or the like, as would be appreciated by one of ordinary skill in the art with the benefit of this disclosure.

In some embodiments, output control system 260 can control various outputs for a corresponding peripheral input device. For instance, output control system 260 may control a number of visual output elements (LEDs, LCDs, and variants and/or combinations thereof), displays, audio outputs (e.g., speakers), haptic output systems, or the like. One of ordinary skill in the art with the benefit of this disclosure would appreciate the many modifications, variations, and alternative embodiments thereof.

Although certain systems may not be expressly discussed, they should be considered as part of system 200, as would be understood by one of ordinary skill in the art. For example, system 200 may include a bus system to transfer power and/or data to and from the different systems therein.

It should be appreciated that system 200 is illustrative and that variations and modifications are possible. System 200 can have other capabilities not specifically described herein. Further, while system 200 is described with reference to particular blocks, it is to be understood that these blocks are defined for convenience of description and are not intended to imply a particular physical arrangement of component parts. Further, the various system blocks need not correspond to physically distinct components. Blocks can be configured to perform various operations, e.g., by programming a processor or providing appropriate control circuitry, and various blocks might or might not be reconfigurable depending on how the initial configuration is obtained.

Embodiments of the present invention can be realized in a variety of apparatuses including electronic devices (e.g., peripheral devices) implemented using any combination of circuitry and software. Furthermore, aspects and/or portions of system 200 may be combined with or operated by other sub-systems as required by design. For example, input detection module 250 and/or memory 220 may operate within processor(s) 210 instead of functioning as a separate entity. In addition, the inventive concepts described herein can also be applied to any peripheral device. Further, system 200 can be applied to any of the input devices described in the embodiments herein, whether explicitly, referentially, or tacitly described (e.g., would have been known to be applicable to a particular input device by one of ordinary skill in the art). The foregoing embodiments are not intended to be limiting and those of ordinary skill in the art with the benefit of this disclosure would appreciate the myriad applications and possibilities.

### System for Operating a Host Computing Device

FIG. 3 is a simplified block diagram of a computing device 300, according to certain embodiments. Computing device 300 can implement some or all functions, behaviors, and/or capabilities described above that would use electronic storage or processing, as well as other functions, behaviors, or capabilities not expressly described. Computing device 300 includes a processing subsystem (processor(s)) 302, a storage subsystem 306, user interfaces 314, 316, and a communication interface 312. Computing device 300 can also include other components (not explicitly shown) such as a battery, power controllers, and other components operable to provide various enhanced capabilities. In various embodiments, computing device 300 can be implemented in a host computing device, such as a desktop 110 or laptop computer, mobile device (e.g., tablet computer, smart phone, mobile phone), tablet computer, wearable device, media device, or the like, in peripheral devices (e.g., keyboards, etc.) in certain implementations.

Processor(s) 302 can include MCU(s), micro-processors, application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or electronic units designed to perform a function or combination of methods, functions, etc., described throughout this disclosure.

Storage subsystem 306 can be implemented using a local storage and/or removable storage medium, e.g., using disk, flash memory (e.g., secure digital card, universal serial bus flash drive), or any other non-transitory storage medium, or a combination of media, and can include volatile and/or non-volatile storage media. Local storage can include a memory subsystem 308 including random access memory (RAM) 318 such as dynamic RAM (DRAM), static RAM (SRAM), synchronous dynamic RAM (e.g., DDR), or battery backed up RAM or read-only memory (ROM) 320, or a file storage subsystem 310 that may include one or more code modules. In some embodiments, storage subsystem 306 can store one or more applications and/or operating system programs to be executed by processing subsystem 302, including programs to implement some or all operations described above that would be performed using a computer. For example, storage subsystem 306 can store one or more code modules for implementing one or more method steps described herein.

A firmware and/or software implementation may be implemented with modules (e.g., procedures, functions, and so on). A machine-readable medium tangibly embodying instructions may be used in implementing methodologies described herein. Code modules (e.g., instructions stored in memory) may be implemented within a processor or external to the processor. As used herein, the term “memory” refers to a type of long term, short term, volatile, nonvolatile, or other storage medium and is not to be limited to any particular type of memory or number of memories or type of media upon which memory is stored.

Moreover, the term “storage medium” or “storage device” may represent one or more memories for storing data, including read only memory (ROM), RAM, magnetic RAM, core memory, magnetic disk storage mediums, optical storage mediums, flash memory devices and/or other machine readable mediums for storing information. The term “machine-readable medium” includes, but is not limited to, portable or fixed storage devices, optical storage devices, wireless channels, and/or various other storage mediums capable of storing instruction(s) and/or data.

Furthermore, embodiments may be implemented by hardware, software, scripting languages, firmware, middleware, microcode, hardware description languages, and/or any combination thereof. When implemented in software, firmware, middleware, scripting language, and/or microcode, program code or code segments to perform tasks may be stored in a machine readable medium such as a storage medium. A code segment (e.g., code module) or machine-executable instruction may represent a procedure, a function, a subprogram, a program, a routine, a subroutine, a module, a software package, a script, a class, or a combination of instructions, data structures, and/or program statements. A code segment may be coupled to another code segment or a hardware circuit by passing and/or receiving information, data, arguments, parameters, and/or memory contents. Information, arguments, parameters, data, etc. may be passed, forwarded, or transmitted by suitable means including memory sharing, message passing, token passing, network transmission, etc. These descriptions of software, firmware, storage mediums, etc., apply to systems 200 and 300, as well as any other implementations within the wide purview of the present disclosure. In some embodiments, aspects of the invention (e.g., surface classification) may be performed by software stored in storage subsystem 306, stored in memory 220 of a peripheral device, or both. One of ordinary skill in the art with the benefit of this disclosure would appreciate the many modifications, variations, and alternative embodiments thereof.

Implementation of the techniques, blocks, steps and means described throughout the present disclosure may be done in various ways. For example, these techniques, blocks, steps and means may be implemented in hardware, software, or a combination thereof. For a hardware implementation, the processing units may be implemented within one or more ASICs, DSPs, DSPDs, PLDs, FPGAs, processors, controllers, micro-controllers, microprocessors, other electronic units designed to perform the functions described above, and/or a combination thereof

Each code module may comprise sets of instructions (codes) embodied on a computer-readable medium that directs a processor of a computing device 110 to perform corresponding actions. The instructions may be configured to run in sequential order, in parallel (such as under different processing threads), or in a combination thereof After loading a code module on a general purpose computer system, the general purpose computer is transformed into a special purpose computer system.

Computer programs incorporating various features described herein (e.g., in one or more code modules) may be encoded and stored on various computer readable storage media. Computer readable media encoded with the program code may be packaged with a compatible electronic device, or the program code may be provided separately from electronic devices (e.g., via Internet download or as a separately packaged computer readable storage medium). Storage subsystem 306 can also store information useful for establishing network connections using the communication interface 312.

Computer system 300 may include user interface input devices 314 elements (e.g., webcam, near infrared (IR) image sensor, touch pad, touch screen, scroll wheel, click wheel, dial, button, switch, keypad, microphone, etc.), as well as user interface output devices 316 (e.g., video screen, indicator lights, speakers, headphone jacks, virtual- or augmented-reality display, etc.), together with supporting electronics (e.g., digital to analog or analog to digital converters, signal processors, etc.). A user can operate input devices of user interface 314 to invoke the functionality of computing device 300 and can view and/or hear output from computing device 300 via output devices of user interface 316.

Processing subsystem 302 can be implemented as one or more processors (e.g., integrated circuits, one or more single core or multi core microprocessors, microcontrollers, central processing unit, graphics processing unit, etc.). In operation, processing subsystem 302 can control the operation of computing device 300. In some embodiments, processing subsystem 302 can execute a variety of programs in response to program code and can maintain multiple concurrently executing programs or processes. At a given time, some or all of a program code to be executed can reside in processing subsystem 302 and/or in storage media, such as storage subsystem 304. Through programming, processing subsystem 302 can provide various functionality for computing device 300. Processing subsystem 302 can also execute other programs to control other functions of computing device 300, including programs that may be stored in storage subsystem 304.

Communication interface (also referred to as network interface) 312 can provide voice and/or data communication capability for computing device 300. In some embodiments, communication interface 312 can include radio frequency (RF) transceiver components for accessing wireless data networks (e.g., Wi-Fi network; 3G, 4G/LTE; etc.), mobile communication technologies, components for short range wireless communication (e.g., using Bluetooth communication standards, NFC, etc.), other components, or combinations of technologies. In some embodiments, communication interface 312 can provide wired connectivity (e.g., universal serial bus (USB), Ethernet, universal asynchronous receiver/transmitter, etc.) in addition to, or in lieu of, a wireless interface. Communication interface 312 can be implemented using a combination of hardware (e.g., driver circuits, antennas, modulators/demodulators, encoders/decoders, and other analog and/or digital signal processing circuits) and software components. In some embodiments, communication interface 312 can support multiple communication channels concurrently.

User interface input devices 314 may include any suitable computer peripheral device (e.g., computer mouse 140, keyboard 130, webcam 160, gaming controller, remote control, stylus device, etc.), as would be appreciated by one of ordinary skill in the art with the benefit of this disclosure. User interface output devices 316 can include display devices (e.g., a monitor, television, projection device, etc.), audio devices (e.g., speakers, microphones), haptic devices, etc. Note that user interface input and output devices are shown to be a part of system 300 as an integrated system. In some cases, such as in laptop computers, this may be the case as keyboards and input elements as well as a display and output elements are integrated on the same host computing device. In some cases, the input and output devices may be separate from system 300, as shown in FIG. 1. One of ordinary skill in the art with the benefit of this disclosure would appreciate the many modifications, variations, and alternative embodiments thereof.

It will be appreciated that computing device 300 is illustrative and that variations and modifications are possible. A host computing device can have various functionality not specifically described (e.g., voice communication via cellular telephone networks) and can include components appropriate to such functionality. While the computing device 300 is described with reference to particular blocks, it is to be understood that these blocks are defined for convenience of description and are not intended to imply a particular physical arrangement of component parts. For example, processing subsystem 302, storage subsystem 306, user interfaces 314, 316, and communications interface 312 can be in one device or distributed among multiple devices. Further, the blocks need not correspond to physically distinct components. Blocks can be configured to perform various operations, e.g., by programming a processor or providing appropriate control circuitry, and various blocks might or might not be reconfigurable depending on how an initial configuration is obtained. Embodiments of the present invention can be realized in a variety of apparatus including electronic devices implemented using a combination of circuitry and software. Host computing devices or even peripheral devices described herein can be implemented using system 300.

### Detecting User Posture and Identifying Posture Types

As described above, aspects of the invention can analyze a user's posture to determine if the posture is good or bad, and make recommendations for the user to improve their posture. Recommendations can be made in real-time, periodically, in summary format (e.g., daily or weekly report of posture performance over intervals), or the like. A user's posture may be classified as “good” (e.g., healthy) if the user's upper body is sufficiently aligned with a predetermined reference—typically a reference line. In some embodiments, the reference line can be a (virtual) vertical line (also referred to herein as a vertical axis 410) determined via a webcam or other imaging system or sensor suite, and may further utilize other sensor devices (e.g., IMU) to determine a reference vertical line in a user-inhabited 3D physical space. In certain embodiments, the vertical axis relative to the user may be acquired in conjunction with a user inquiry. For example, a user may be prompted via audio or video (e.g., graphical user interface (“GUI”)) to sit on a chair with good posture (e.g., instruct a user to sit as vertically positioned as they can while they are scanned/detected). The sensor data corresponding to the user with good posture (e.g., image data, force data, etc.) can be used as reference data to determine the vertical axis relative to the user.

In some embodiments, a horizontal axis can be used in conjunction with the vertical axis to help determine a posture of a user. In some cases, the horizontal axis can be used to determine if a user's shoulders are in alignment and not slouching or leaning too much one way or another. In certain embodiments, a classification of a user's posture can be determine with or without the use of a horizontal axis. A horizontal axis can be determined via a webcam or other imaging system or sensor suite, and may further utilize other sensor devices (e.g., IMU) to determine a reference horizontal line in a user-inhabited 3D physical space. In certain embodiments, the horizontal axis relative to the user may be acquired in conjunction with a user inquiry. For example, a user may be prompted via audio or video (e.g., graphical user interface (“GUI”)) to sit on a chair with good posture (e.g., instruct a user to sit as vertically positioned with even shoulder position as they can while they are scanned/detected). The sensor data corresponding to the user with good posture (e.g., image data, force data, etc.) can be used as reference data to determine the horizontal axis relative to the user.

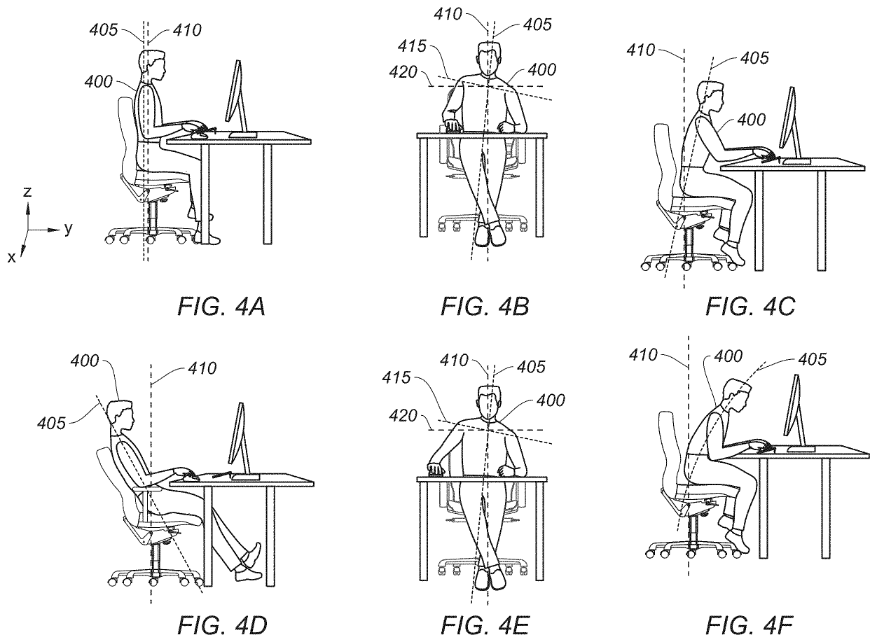

FIGS. 4A-4F show examples of different posture types that can be used by certain embodiments. FIG. 4A shows an example of good ergonomic posture with user 400 sitting at a desk or workstation with their back/spine (corresponding to line 405) aligned with vertical axis 410. Vertical axis 410 is shown in the figures as a line in one dimension, however it should be understood that vertical axis 410 is typically vertical from any radius angle perpendicular to vertical axis 410 (e.g., 360 degrees around the user). As one example, Cartesian coordinates axes x, y, and z are shown relative to the user with vertical axis 410 being co-linear with the z-axis. Generally, if the user's spine is aligned with vertical axis 410, their posture is generally deemed to be good or ideal, and if the user's spine is misaligned with and/or deviates from vertical axis 410 by a threshold amount, as can be the case in a number of body postures, the user's posture is typically deemed bad or non-ideal.

FIG. 4B shows an example of bad posture with the user sitting at a work station with crossed feet, shoulders forward, and/or they are leaning on one elbow or forearm. In this case, the user's posture can be classified as having asymmetry. Note that the user's spine 405 is not aligned with vertical axis 410. Furthermore, the user's shoulders (corresponding to line 415) are not aligned with horizontal axis 420. In some cases, other characteristics of the user can be used to characterize a user as having asymmetrical posture, as would be appreciated by one of ordinary skill in the art with the benefit of this disclosure.

FIG. 4C shows an example of bad posture with the user sitting at a work station with the display configured too far forward, the bottom of the user's feet (or the user's toes) pointed backwards rather than planted on the floor, and the desk is too low such that the user is leaned forward to accommodate the arrangement. In this case, the user's posture can be classified as configured forward. Note that the user's spine 405 is not aligned with vertical axis 410. In some cases, other characteristics can be used to characterize a user as having forward posture, as would be appreciated by one of ordinary skill in the art with the benefit of this disclosure.

FIG. 4D shows an example of bad posture with the user sitting at a work station and leaning backwards with the bottom of the user's heels (or the user's toes) pointed forward rather than planted on the ground, and the user's shoulders being supported by the user's elbows. In this case, the user's posture can be classified as configured backwards. Note that the user's spine 405 is not aligned with vertical axis 410. In some cases, other characteristics can be used to characterize a user as having backward posture, as would be appreciated by one of ordinary skill in the art with the benefit of this disclosure.

FIG. 4E shows an example of bad posture with the user sitting at a work station and leaning with at least one elbow to the side, which can be due to the desk being too high, the computer mouse too far from the user, or the like. In this case, the user's posture can be classified as “elbows to the side” or other suitable name. Note that the user's spine 405 is not aligned with vertical axis 410. In some cases, the user's shoulders 415 may not be aligned with horizontal axis 420. Other characteristics can be used to characterize a user as having backward posture, as would be appreciated by one of ordinary skill in the art with the benefit of this disclosure.

FIG. 4F shows an example of bad posture with the user sitting at a work station and slouching with rounded shoulders, which can be due to the desk being too low, the keyboard being configured too far forward, or the like. In this case, the user's posture can be classified as “rounded shoulders” or other suitable name. Note that the user's spine 405 is not aligned with vertical axis 410. In some cases, other characteristics can be used to characterize a user as having backward posture, as would be appreciated by one of ordinary skill in the art with the benefit of this disclosure.

Some embodiments may deviate from the classifications described above. For example, other types of “bad” posture classifications can be used that may relate to a user's back/spine position or shoulder position relative to a vertical or horizontal axis, respectively. In some aspects, “good” posture may depend on an activity. More specifically, the activity may correspond to a software application that the user is interacting with. If the user is doing productivity type work (e.g., word processing, spreadsheets, etc.), then the posture of FIG. 4A may be preferred. With certain activities, such as video calls, movies, or other computer-related interactions that may not necessarily call for the posture shown in FIG. 4A, a more relaxed posture (e.g., FIG. 4D) may be acceptable and may not prompt the system to take corrective action. However, even in a relaxed position, there may be user posture adjustment that can be made for improved ergonomic support for the user's back, shoulders, etc., such that posture recommendations can still be made for alternative good postures corresponding to particular activities. One of ordinary skill in the art with the benefit of this disclosure would appreciate the many modifications, variations, and alternative embodiments thereof.

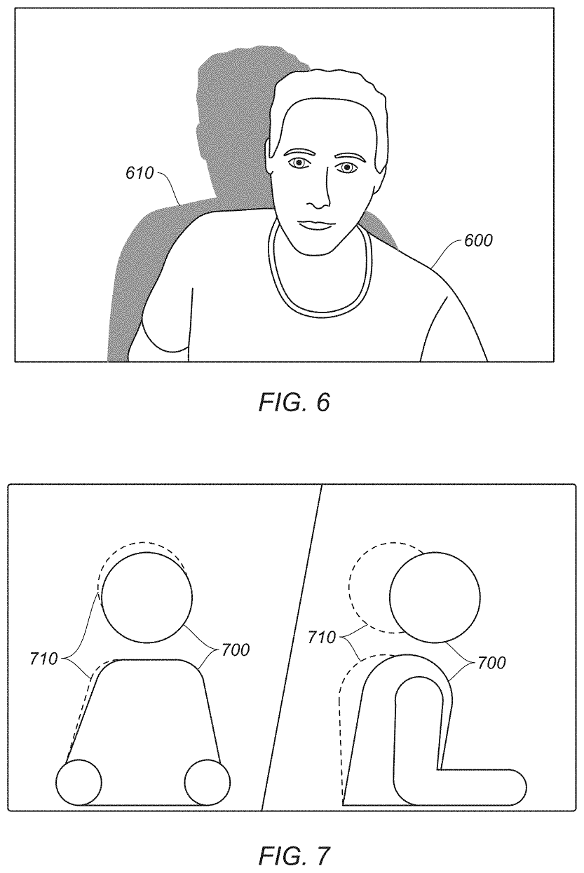

The system can analyze the user's posture via any suitable sensor suite that may comprise of images sensor(s) (e.g., webcam, near infrared image sensor, 3D camera such as time-of-flight, structured light, stereoscopic, dual pixel, images sensors, deep learning based monocular depth estimation), pressure sensors (e.g., chair), inertial measurement unit (IMU) (e.g., on clothing, furniture, wearables, etc.), motion sensors, or other suitable sensor type and/or combination thereof. In some aspects, recommendations may include video-based feedback that shows an image of the user and posture, and an underlying reference images showing how the user should be sitting in order to have good posture, as shown and described below with respect to FIG. 6. The user can adjust their posture in real-time until the user's image and the reference image are sufficiently aligned. In some cases, feedback can be given in the form of an avatar representation of the user's posture in comparison to a good posture reference, as shown and described below with respect to FIG. 7. Any suitable method of recommendation can be used as would be appreciated by one of ordinary skill in the art with the benefit of this disclosure. As noted above, the user's activity may affect the recommendation as different activities may warrant different postures or parameters of what constitutes “good” posture.

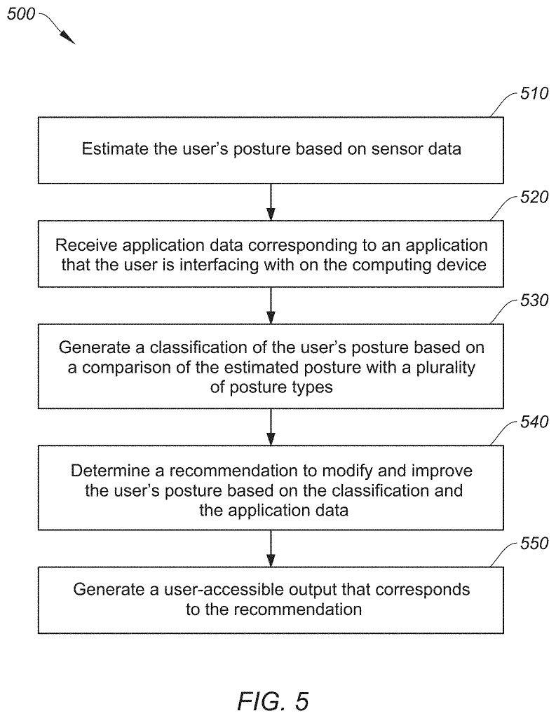

FIG. 5 is a simplified flow chart showing aspects of a method 500 for monitoring a recommending posture to a user, according to certain embodiments. Method 500 can be performed by processing logic that may comprise hardware (circuitry, dedicated logic, etc.), software operating on appropriate hardware (such as a general purpose computing system or a dedicated machine), firmware (embedded software), or any combination thereof. In certain embodiments, method 500 can be performed by aspects of system 300 (e.g., a host computing device), system 200 (e.g., one or more computer peripheral devices), or a combination thereof, as described throughout this disclosure and as would be appreciated by one of ordinary skill in the art with the benefit of this disclosure. By way of example, method 500 may be performed by processor(s) 302 of system 300 (e.g., computer 110) using webcam 150 as the sensor suite to detect the user's posture and display 120 to provide visual feedback of the user's posture to the user, according to certain embodiments.

At operation 510, method 500 can include estimating a user's posture based on sensor data from a sensor suite, according to certain embodiments. The sensor suite can include one or more of an optical sensor from an optical device (e.g., webcam) configured to generate optical sensor data corresponding to the 3D orientation of the user's body (e.g., video or images showing the user's position and orientation (“pose”); pressure sensor(s) from a piece of furniture (e.g., sensor-outfitted chair) configured to generate force sensor data corresponding to the 3D orientation of the user's body (e.g., forces detected by multiple sensors on a chair can be used to determine how a user's weight is distributed on the chair and ultimately aspects of the user's posture based on differences and/or asymmetries in weight distribution over the chair); motion sensor(s) from a wearable device configured to generate motion sensor data corresponding to the 3D orientation of the user's body; or other suitable sensor device that can be used to glean information about a user's posture, as would be appreciated by one of ordinary skill in the art with the benefit of this disclosure.

In some aspects, method 500 can further include determining a vertical axis and/or a horizontal axis relative to the user, where estimating the user's posture is further based on the detected orientation of the user's body as compared to the vertical axis and/or horizontal axis relative to the user. In some embodiments, a vertical and/or horizontal axes can be determined automatically via image data, IMU data, computer vision methods to determine vertical and/or horizontal from 2D images taken by webcam, magnetic angle gauges that are configured to determine the horizontal and vertical, or other process. In some aspects, the vertical axis and/or horizontal axes can be determined by asking a user to sit more towards vertical and in a position and orientation that would be considered good (healthy) posture. The system can detect the person's posture via the sensor suite and use it to establish the vertical and/or horizontal axes.

At operation 520, method 500 can include receiving application data corresponding to an application that the user is interfacing with on the computing device, according to certain embodiments. The application data may correspond to at least one of a list applications including a video gaming application (e.g., FPS, RTS, eSports, etc.), a productivity application (e.g., word processing, spreadsheets, presentation, coding, etc.), a video conferencing application (e.g., Zoom®, WebEx®, etc.), collaboration applications, or any other software-based application, typically with a graphical user interface for user interaction.

At operation 530, method 500 can include generating a classification of the user's posture based on a comparison of the estimated posture with a plurality of posture types, according to certain embodiments. The plurality of posture types may include good posture and bad posture, where the bad pasture can include at least one of asymmetry, forward leaning, backward leaning, side leaning, elbows to the side, and rounded shoulders, as shown and described above with respect to FIGS. 4A-4F. Other posture types can be used and multiple versions of good posture can be used, which can be application dependent. Some additional posture types may include a lateral bending of a user's head, rotation of the user's head around a neck axis, flexion and extension of the user's neck, or the like. As noted above, a good posture for productivity may differ for an allowed good posture for collaboration or video conferencing applications, as described above and as would be appreciated by one of ordinary skill in the art with the benefit of this disclosure. The classification may correspond to a best matching of the user's posture with the plurality of postures based on the comparison. In some cases, the user's posture may have elements of multiple posture types. For example, the user may be slightly leaned to the side and leaning backwards. In such cases, the classification may correspond to the best fit, e.g., the posture type that the user's posture is more closely matched with. In some aspects, the classification may be a hybrid of posture types. For example, the user's posture may be classified as forward leaning with rounded shoulders. In such cases, the subsequent recommendation may factor in single or hybrid classifications when suggesting corrective posture adjustments to the user.

At operation 540, method 500 can include determining a recommendation to modify and improve the user's posture based on the classification and the application data, according to certain embodiments. In some embodiments, determining the recommendation to modify and improve the user's posture can include determining body posture modifications to get the user to have good posture while operating the given application. For instance, if a user is leaning forward, the determined recommendation may call for the user to move to an upright position and preferably such that the user's back/spine is aligned with or substantially aligned with (e.g., within 5 degrees) of the vertical axis. In some cases, the recommendation may call for the user to sit with straight shoulders such that the user's shoulders are aligned with or substantially aligned with (e.g., within 5 degrees of) the horizontal axis. In some cases, alignment tolerance with respect to the vertical (and/or horizontal) axis may depend on a particular joint. For instance, in some implementations a user's back may lean up to 10 degrees, whereas an angle between the user's leg and their trunk (back/core) may vary by as much as 25 degrees. Other tolerances are possible, as would be appreciated by one of ordinary skill in the art with the benefit of this disclosure.

At operation 550, method 500 can include generating a user-accessible output that corresponds to the recommendation, according to certain embodiments. In some aspects, the user-accessible output includes a video underlay showing a user's target posture (e.g., showing a good posture in silhouette) so that the user can visually see how their current actual posture can be adjusted to better match the good posture, as shown and described below with respect to FIG. 6. In some aspects, the user-accessible output includes an animated avatar showing a user's target posture versus the user's current actual posture, as shown and described below with respect to FIG. 7. Alternatively or additionally, a recommendation may be made to the user in response to determining that the user has bad posture. For instance, if a user is determined to be leaning back, one or more questions or observations can be presented to the user, such as a comment that the user's may be sliding in their chair, or a recommendation that the user take a break or stretch if their posture is progressively getting worse. One of ordinary skill in the art with the benefit of this disclosure would appreciate the many modifications, variations, and alternative embodiments thereof.

In certain embodiments, method 500 can also include determining a posture quality score that corresponds to a good posture versus bad posture ratio over a given period of time, wherein generating the user-accessible output further includes generating a graphical representation of the posture quality score. For example, if a user exhibits good posture for a few minutes, but exhibits bad posture (e.g., similar to any of FIGS. 4B-4F) for several hours, their posture quality score may be low. Conversely, if the user has good posture 95% of the time, but bad posture 5% of the time, then the user's posture quality score may be high.

In certain embodiments, the posture quality score may operate under the principle of losing points over time. For example, a posture quality score may start at 1000 and if the user's posture is subsequently bad for a period of time, the user misses a scheduled break from sitting, etc., the posture quality score may be reduced. One possible scheme for a scoring system may include calculating a score as follows: (1) a user's posture is calculated at a particular interval (e.g., 1 frame per second (fps)); (2) at a longer interval (e.g., 1 min) if the user's posture is classified at a particular posture more than 50% then that posture is tagged for the longer interval (e.g., recorded as the posture for that interval, despite that other postures may have occurred less frequently or at less duration), otherwise the longer interval may be tagged as having good posture; (3) over an even longer interval (e.g., 10 min) a percentage of good posture over bad posture (e.g., any posture other than good posture) may be calculated. In some cases, an “empty bucket” can be used to an average time before breaks. In some aspects, the formula to calculate the score can be two sigmoid functions, one for the user's posture and one for the breaks. One of ordinary skill in the art with the benefit of this disclosure would appreciate the many modifications, variations, and alternative embodiments thereof

It should be appreciated that the specific steps illustrated in FIG. 5 provide a particular method 500 for monitoring a recommending posture to a user, according to certain embodiments. Other sequences of steps may also be performed according to alternative embodiments. Furthermore, additional steps may be added or removed depending on the particular applications. Any combination of changes can be used and one of ordinary skill in the art with the benefit of this disclosure would understand the many variations, modifications, and alternative embodiments thereof.

It should be noted that aspects of the invention may focus on providing feedback about a user's posture in a non-intrusive manner, such as giving video feedback in the background rather than a foreground of a display, along the edges of the display, at a preferred frequency (e.g., every hour, once per day, once per 10 minutes, etc.), or the like. Alternatively or additionally, audio cues can be used to provide posture feedback to the user. In some cases, the feedback can be in real-time (e.g., as shown in FIGS. 6 and 7) and/or feedback can be aggregated over time via charts, graphs, dashboards, etc., showing posture metrics to help a user achieve posture goals. For example, a graph may show that a user has good posture 15% of time, which is an increase of 3% week to week, which may be a positive indicator and motivator to the user to progressively increasing their good posture percentage over time.

Given the wide variety of body types including a user's height, weight, size, proportions, etc., a universal application of a single classification system may work better for some users than others some embodiments, a deep learning classifier algorithm can be used to classify a user's posture. For example, a deep learning classifier may estimate a user's posture (e.g., based on sensor data) and determine a 3D skeleton of the user's orientation and pose, which can be classified based on symmetry, a comparison with known posture types, and in some cases an application that the user is interfacing with. The system can determine how long a user orients their posture according to the various known posture types and can learn user tendencies over time. For instance, once user may tend to lean back much more than another user. Thus, some aspects may incorporate multiple user profiles where each profile incorporates machine learning to better adapt to user profile tendencies and can better provide feedback to correct the user's posture, to proactively remind a user they tend to lean a certain way so they are aware and do not deviate from a good posture, and also in some cases provide the feedback in a preferred format (e.g., underlay, avatar, audio only, summary dashboards) and frequency (e.g., continuously, periodically, etc.). This process of classifying and recommending can be iterative, such that a deep learning algorithm be able to improve characterization accuracy, progressively fine tune feedback, and better accommodate user preferences, as would be appreciated by one of ordinary skill in the art with the benefit of this disclosure. In some cases, based on the classification over a period of time (e.g., seconds, hours, days, weeks, etc.), feedback may include a recommendation for dynamic posture (the user should not stay static too long and may have slight changes in pose), recommendations against repetitive movement that stress joints, and overall monitoring that provides instant notifications, periodic summaries (e.g., daily, weekly, monthly, etc.), and the like.

In some embodiments, a classification may be determined as a probability. For example, an estimated posture of a user may be determined to be classified as an 85% chance to be good, 10% asymmetrical, and 5% slouching with rounded shoulders. A posture classification may be based on the posture type with the highest estimated percentage (e.g., current, over time, etc.). In some cases, a user may determine that a classified posture is not accurate all of the time. In such cases, the user may provide feedback to the system (e.g., via voice, keyboard, selection on a GUI via a computer mouse, etc.) to correct and train/improve the classification of some or all of the various posture types.

FIG. 6 shows a graphical user interface showing a video underlay that provides a visual reference of good posture as compared to a user's current posture, according to certain embodiments. User 600 is shown in a video conferencing application with a silhouetted (shadow) underlay 610 that represents how the user should be positioned to achieve good posture. The underlay may be less obtrusive and can provide an easy reference for the user to adjust their posture accordingly. In some cases, it may not be preferable to provide a shadow underlay if the video is shared between multiple users. As such, some implementations may cause the system to take control of the camera and split the video feed such that one feed is applied to the video conferencing application and a second feed is used to provide posture feedback as shown, for example, in FIG. 6.

FIG. 7 shows a graphical user interface showing an avatar-based representation 700 of a user and a visual reference 710 of good posture for comparison, according to certain embodiments. User 700 can be shown can be shown in a front view and/or side view to represent a user's posture. In some cases, only a front view or side view can be used, or multiple views may be provided. User 700 is shown with a forward posture, which is more easily seen in the side view than the front view. The avatar-based representation can be presented in real-time or periodically (e.g., an aggregate view showing average user position over time versus reference 710). The avatar can be a 2D rendition or a 3D rendition generated by a 3D engine. One of ordinary skill in the art with the benefit of this disclosure would appreciate the many modifications, variations, and alternative embodiments thereof

In certain embodiments, if a user is determined to have bad posture over a particular time (e.g., 1 min, 5 min, etc.), the system may provide a message to the user (e.g., audio and/or video message) to suggest changing or modifying their posture, stretching, or performing other activities that can foster better posture in a user. In some cases, a recommendation (e.g., the graphical/video representations of FIGS. 6-7) may be instantiated after a particular duration, frequency, and/or severity of bad posture. In some cases, the severity of the bad posture (e.g., large deviation between actual posture and “good” posture silhouette) or long term chronically bad posture may inform how recommendations can be provided. For example, a static posture target can be provided (e.g., as shown in FIGS. 6-7) for immediate correction towards good posture. In some cases, a dynamic posture correction can be applied where the guiding graphic (e.g., avatar) prompts the user to make small corrective adjustments that improve the user's overall posture over time, with the goal of slowly moving the user's posture habits towards an ideal “good” posture over a series of incremental steps (e.g., over a period of minutes, hours, days, etc.) rather than (or at times in addition to) a single large movement.

In certain embodiments, audio cues may be provided to a user to improve their posture. In some aspects, the audio cues can be based on the current posture classification of the user. For instance, the audio cue may instruct the user to sit upright, move a particular shoulder up, avoid leaning to one side, move the computer peripherals to a better ergonomic location, sit up higher on a chair, avoid crossing legs, be mindful of tendencies to slouch or lean a certain way after a period of time, etc. In some cases, visual cues may do the same. Besides a graphic showing an ideal, as shown in FIGS. 6 and 7, a dynamic graphic may be provided that shows the user how they can move to achieve a better posture. For instance, a graphic may show a user (e.g., image of a person or avatar) moving from a leaned back position to an upright position that embodiments good posture. In some aspects, feedback may be partially dependent on an application that the user is interacting with. For example, posture related feedback may be more involved (e.g., more frequent, recommend bigger changes in posture, etc.) when the user is using software applications that typically call for better posture (e.g., productivity apps, gaming apps, etc.) and less involved for applications where good posture may not be as important (e.g., video conferencing applications). In some cases, the software, user, or a combination thereof, may define which applications should have a more aggressive posture detection/recommendation schema than other applications. One of ordinary skill in the art with the benefit of this disclosure would appreciate the many modifications, variations, and alternative embodiments thereof

Alternatively or additionally, haptics can be used to apply user feedback. For instance, a smart chair, keyboard, mouse, headset, HMD, or other device in contact with the user may vibrate when their posture is determined to be bad. In some aspects, the intensity of the haptic sensation may correspond to how far off of a “good” posture the user is, how long the user has had bad posture over a period of time, if the user is regressing in posture habits rather than improving, or the like, as would be appreciated by one of ordinary skill in the art with the benefit of this disclosure.

In further embodiments, unwanted non-posture-related habits may be incorporated into the various posture training implementations described herein. For instance, nail biting, fidgeting during a conference call, reminders to the user that they are not muted if it appears they are directed a conversation in another direction other than the display device, or the like, can leverage the technologies described herein. For example, audio, visual, and/or haptics can be used to inform a user when they are doing the unwanted habit. Feedback can be instantaneous (e.g., Pavlovian) or aggregated and presented in a summary format, as described in the other embodiments above.

In certain embodiments, users may be assigned a user profile. The user profile can run analytics that track and correlate a user's posture with application performance characteristics. For example, user analytics may show that a user types more words per minute on average when they have good posture. In some cases, some analytics may show that a user tends to perform better in gaming applications (e.g., FPS, RTS, eSports, etc.) when they sit forward or more upright. In such cases, a forward leaning posture recommendation may be made when the user is playing certain gaming applications, and a conventional good posture (e.g., FIG. 4A) may be made when the user is interfacing with other applications (e.g., productivity applications). That is, some embodiments may have more than one “good” posture, with one or more acceptable postures depending on the user's activity. One of ordinary skill in the art with the benefit of this disclosure would appreciate the many modifications, variations, and alternative embodiments thereof. Generally, a healthy or “good” posture, as applied to any of the embodiments contemplated throughout this disclosure, can be considered a posture that distributes the user's weight over a variety of tissues (e.g., bones, muscles, connective tissues, joints, etc.) without overloading them (or minimizing any overloading) over time. Typically, this good posture may correspond to FIG. 4A. A dynamic posture can be promoted as well, which has the user change their posture (e.g., shift, stretch, stand, walk around, etc.) over time (e.g., at an interval — every 10 minutes, whenever poor posture is detected after a period of good posture, etc.), or the like. One of ordinary skill in the art with the benefit of this disclosure would appreciate the many modifications, variations, and alternative embodiments thereof

Most embodiments utilize at least one network that would be familiar to those skilled in the art for supporting communications using any of a variety of commercially available protocols, such as TCP/IP, UDP, OSI, FTP, UPnP, NFS, CIFS, and the like. The network can be, for example, a local area network, a wide-area network, a virtual private network, the Internet, an intranet, an extranet, a public switched telephone network, an infrared network, a wireless network, and any combination thereof.

In embodiments utilizing a network server as the operation server or the security server, the network server can run any of a variety of server or mid-tier applications, including HTTP servers, FTP servers, CGI servers, data servers, Java servers, and business application servers. The server(s) also may be capable of executing programs or scripts in response to requests from user devices, such as by executing one or more applications that may be implemented as one or more scripts or programs written in any programming language, including but not limited to Java®, JavaScript, C, C# or C++, or any scripting language, such as Perl, Python or TCL, as well as combinations thereof. The server(s) may also include database servers, including without limitation those commercially available from Oracle Microsoft Sybase® and IBM®.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a non-transitory computer readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services or other elements located within at least one working memory device, including an operating system and application programs, such as a client application or browser. It should be appreciated that alternate embodiments may have numerous variations from that described above. For example, customized hardware might also be used and/or particular elements might be implemented in hardware, software (including portable software, such as applets) or both. Further, connections to other computing devices such as network input/output devices may be employed.

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. The various embodiments illustrated and described are provided merely as examples to illustrate various features of the claims. However, features shown and described with respect to any given embodiment are not necessarily limited to the associated embodiment and may be used or combined with other embodiments that are shown and described. Further, the claims are not intended to be limited by any one example embodiment.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Although the present disclosure provides certain example embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multi-purpose microprocessor-based computer systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some embodiments. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A system for evaluating a posture of a user operating a computing device, the system comprising:
one or more processors;
a sensor suite configured to generate sensor data corresponding to a three-dimensional (3D) orientation of a user's body; and
one or more machine-readable, non-transitory storage mediums that include instructions configured to cause the one or more processors to perform operations including:
estimating the user's posture based on the sensor data from the sensor suite;
receiving application data corresponding to an application that the user is interfacing with on the computing device;
generating a classification of the user's posture based on a comparison of the estimated posture with a plurality of posture types;
determining a recommendation to modify and improve the user's posture based on the classification and the application data; and
generating a user-accessible output that corresponds to the recommendation.

2. The system of claim 1 wherein the instructions are further configured to cause the one or more processors to perform operations including:
determining a vertical axis relative to the user, where estimating the user's posture is further based on the vertical axis relative to the user.

3. The system of claim 1 wherein the sensor suite includes at least one of:
an optical sensor from an optical device configured to generate optical sensor data corresponding to the 3D orientation of the user's body;
a pressure sensor from a piece of furniture configured to generate force sensor data corresponding to the 3D orientation of the user's body; or
a motion sensor from a wearable device configured to generate motion sensor data corresponding to the 3D orientation of the user's body.

4. The system of claim 1 wherein the application data corresponds to at least one of a list applications including:
a video gaming application on the computing device;
a productivity application on the computing device; or
a video conferencing application on the computing device.

5. The system of claim 1 wherein the plurality of posture types includes:
good posture; and
bad posture, wherein the bad posture is made up of the user having at least one of:
asymmetry;
forward leaning;
backward leaning;
side leaning;
elbows to a side;
lateral bending of the user's head;
rotation of the user's head around a neck axis;
flexion and extension of the user's neck; and
rounded shoulders.

6. The system of claim 5 wherein the determining the recommendation to modify and improve the user's posture includes determining body posture modifications that cause the user to have good posture while operating the given application.

7. The system of claim 1 wherein the user-accessible output includes a video underlay showing a user's target posture juxtaposed against the user's current actual posture.

8. The system of claim 1 wherein the user-accessible output includes an animated 2D or 3D avatar showing a user's target posture versus the user's current actual posture.

9. The system of claim 1 wherein the instructions are further configured to cause the one or more processors to perform operations including:
determining a posture quality score that corresponds to a good posture versus bad posture ratio or a bad posture versus good posture ratio over a given period of time,
wherein generating the user-accessible output further includes generating a graphical representation of the posture quality score.

10. A computer-implemented method comprising:
receiving sensor data from a sensor suite, the sensor data corresponding to a 3D orientation of a user's body;
estimating the user's posture based on the received sensor data;
receiving application data corresponding to an application that the user is interfacing with on the computing device;
generating a classification of the user's posture based on a comparison of the estimated posture with a plurality of posture types;
determining a recommendation to modify and improve the user's posture based on the classification and the application data; and
generating a user-accessible output that corresponds to the recommendation.

11. The computer-implemented method of claim 10 further comprising:
determining a vertical axis relative to the user, wherein estimating the user's posture is further based on the vertical axis relative to the user.

12. The computer-implemented method of claim 10 wherein the sensor suite includes at least one of:
an optical sensor from an optical device configured to generate optical sensor data corresponding to the 3D orientation of the user's body;
a pressure sensor from a piece of furniture configured to generate force sensor data corresponding to the 3D orientation of the user's body; or
a motion sensor from a wearable device configured to generate motion sensor data corresponding to the 3D orientation of the user's body.

13. The computer-implemented method of claim 10 wherein the application data corresponds to at least one of a list applications including:
a video gaming application on the computing device;
a productivity application on the computing device; or
a video conferencing application on the computing device.

14. The computer-implemented method of claim 10 wherein the plurality of posture types includes:
good posture; and
bad posture, wherein the bad posture is made up of at least one of the user having:
asymmetry;
forward leaning;
backward leaning;
side leaning;
elbows to a side;
lateral bending of the user's head;
rotation of the user's head around a neck axis;
flexion and extension of the user's neck; and
rounded shoulders.

15. The computer-implemented method of claim 10 wherein the user-accessible output includes at least one of:
a video underlay showing a user's target posture juxtaposed against the user's current actual posture; or
an animated 2D or 3D avatar showing a user's target posture versus the user's current actual posture.

16. A non-transitory computer-program product tangibly embodied in a machine-readable non-transitory storage medium that includes instructions configured to cause one or more processors to perform operations including:
receiving sensor data from a sensor suite, the sensor data corresponding to a 3D orientation of a user's body;
estimating the user's posture based on the received sensor data;
receiving application data corresponding to an application that the user is interfacing with on the computing device;
generating a classification of the user's posture based on a comparison of the estimated posture with a plurality of posture types;
determining a recommendation to modify and improve the user's posture based on the classification and the application data; and
generating a user-accessible output that corresponds to the recommendation.

17. The non-transitory computer-program product of claim 16 wherein the instructions are further configured to cause one or more processors to perform operations including:
determining a vertical axis relative to the user, wherein estimating the user's posture is further based on the vertical axis relative to the user.

18. The non-transitory computer-program product of claim 16 wherein the sensor suite includes at least one of:
an optical sensor from an optical device configured to generate optical sensor data corresponding to the 3D orientation of the user's body;
a pressure sensor from a piece of furniture configured to generate force sensor data corresponding to the 3D orientation of the user's body; or
a motion sensor from a wearable device configured to generate motion sensor data corresponding to the 3D orientation of the user's body.

19. The non-transitory computer-program product of claim 16 wherein the application data corresponds to at least one of a list applications including:
a video gaming application on the computing device;
a productivity application on the computing device; or
a video conferencing application on the computing device.

20. The non-transitory computer-program product of claim 16 wherein the user-accessible output includes at least one of:
a video underlay showing a user's target posture juxtaposed against the user's current actual posture; or
an animated 2D or 3D avatar showing a user's target posture versus the user's current actual posture.
